(19) United States
(12) Patent Application Publication
(10) Pub. No.: US 2011/0151551 A1
(43) Pub. Date: Jun. 23, 2011

(54) BIOREACTORS

(75) Inventors: SHUJIAN YI, Nashua, NH (US); JEFFREY CARTER, Hopkinton, MA (US); TIEYING JIANG, Shrewsbury, MA (US)

(73) Assignee: GE HEALTHCARE BIO-SCIENCES CORP., PISCATAWAY, NJ (US)

(21) Appl. No.: 12/644,305

(22) Filed: Dec. 22, 2009

(51) Int. Cl. C12M 1/02

(52) U.S. Cl. 435/289.1

(57) ABSTRACT

The present invention relates to improved single-use bioreactors comprising disposable plastic bags for cell cultivation. The invention provides an inflatable bioreactor bag for cell cultivation comprised of a top and a bottom sheet of polymer material that are joined along their edges to form a sealed bag, wherein two opposing edges are formed as clamping edges to allow clamping of the bioreactor bag to a rocker type bioreactor, and wherein the bioreactor bag is provided with a wrinkle preventing structure at each end of the clamping edges. The bag avoids formation of undesired wrinkles or creases which otherwise lead to fatigue of the plastic and eventually fracture.

### TECHNICAL FIELD

The present invention relates to improved single-use bioreactors comprising disposable plastic bags for cell cultivation wherein the bags are designed to avoid formation of undesired wrinkles or creases. These may otherwise lead to fatigue of the plastic and eventually fracture.

### BACKGROUND OF THE INVENTION

The bioprocessing industry has traditionally used stainless steel systems and piping in manufacturing processes for fermentation and cell culture. These devices are designed to be steam sterilized and reused. Cleaning and sterilization are however costly labor-intensive operations. Moreover, the installed cost of these traditional systems with the requisite piping and utilities is often prohibitive. Furthermore, these systems are typically designed for a specific process, and cannot be easily reconfigured for new applications. These limitations have led to adoption of a new approach over the last ten years—that of using plastic, single-use disposable bags and tubing, to replace the usual stainless steel tanks.

In particular bioreactors, traditionally made of stainless steel, have been replaced in many applications by disposable bags which are rocked to provide the necessary aeration and mixing necessary for cell culture. These single-use bags are typically sterile and eliminate the costly and time-consuming steps of cleaning and sterilization. The bags are designed to maintain a sterile environment during operation thereby minimizing the risk of contamination.

Bags containing sterile fluids are used in the bioprocessing industry for formulation, storage, transfer, processing, and transportation. Sterile conditions must be maintained during these operations, and the bags are usually sealed to prevent contamination. Commonly used bags are of the “pillow style,” mainly because these can be manufactured at low cost by seaming together two flexible films of plastic. Current disposable cell culture chambers (bags) are designs of 2-D structures.

When bags are inflated with air and medium as well as fixed onto bioreactors, creases around corner area are formed and these corner creases move back and forth with the rocking motion of bioreactor. Some creases will develop fatigue crazes after more than several thousands of cyclic motions. Media leakage and contamination will eventually occur once fatigue crazes penetrate deeply through every constituent layer of the polymer film of the bag.

US2009/0188211A1 (Xcellerex Inc) describes systems and methods for containing and manipulating fluids, such as those involving collapsible bags and rigid containers. Bag wrinkle removing systems are described comprising pneumatically operable bladders that may modify or change the shape of the collapsible bag in order to prevent formation of folds and wrinkles therein.

### SUMMARY OF THE INVENTION

Formation of corner creases is due to high corner stress of inflated bags. The present invention provides disposable cell culture bags that will prevent or minimize the formation of these creases by addressing the corner stress issue. The strategy is to divert corner stress to other areas of the bag or to reinforce corner area so that the rim would not fold itself. Extra structures on the corners and/or side rims of the bag have been found to form a very smooth contour on the corners (minimal to no creases). As a result of significant reduction or removal of corner creases, these new bags will not have any fatigue failure (cracking, delaminating, leaking) during cell culture process.

Thus, in a first aspect the present invention relates to an inflatable bioreactor bag for cell cultivation comprised of a top and a bottom sheet of polymer material that are joined along their edges to form a sealed bag, wherein two opposing edges are formed as clamping edges to allow clamping of said bioreactor bag to a rocker type bioreactor, and wherein the bioreactor bag is provided with a wrinkle preventing structure at each end of said clamping edges.

In one embodiment, the wrinkle preventing structures is comprised of an edge joint segment that interconnects the clamping edge with an adjacent non-clamping edge, and wherein the edge joint segment connects to the clamping edge and the non clamping edge at an angle exceeding 150°.

In another embodiment, the edge joint segment is a continuous curved joint segment.

In a further embodiment, the edge joint segment is an essentially straight joint segment.

In yet a further embodiment, each wrinkle preventing structure extends more than 2% and less than 15% of the adjacent non reinforced edge, to allow unrestricted motion of the intermediate section.

Preferably, one or more of the wrinkle preventing structures is comprised of a reinforced section of an adjacent non-clamping edge. The reinforced section may be continuously interconnected with the clamping edge at an angle.

Thus, the bioreactor bag preferably comprises extra structures in the corner areas of the bag in such a way that the inner volume of the bag contains no sharp corners (angles).

### BRIEF DESCRIPTION OF THE DRAWINGS

FIG. 1 is a schematic view of a disposable bag having triangular structures in the corners, i.e. the bag is shaped like an octagonal.

FIG. 2 is a schematic view of a disposable bag having pseudoround corners.

FIG. 3 is a schematic view of a disposable bag having reinforced rims.

FIG. 4 is a schematic view of a disposable bag having reinforced rims integrated with the reinforced rims on conventional bags for cell cultivation.

### DETAILED DESCRIPTION OF THE INVENTION

The present invention is an improvement of a bioreactor that consists of a pre-sterilized flexible plastic bag in which cells are cultivated. The bag is partially filled with growth media and the remainder of the bag is continuously purged with air or other oxygen-rich gas. The bag is placed on a platform that can be rocked to and fro. The rocking motion promotes wave formation in the bag, which provides liquid mixing and enhances oxygen transfer from the headspace gas to the liquid phase where it is essential for cell growth and metabolism. The air in the bag performs several functions: 1) allows the formation of surface waves promoting oxygen transfer; 2) continually provides fresh oxygen into the bag and sweeps out gaseous metabolic products and 3) inflates the bag to a rigid form which reduces foam formation and promotes liquid mixing.

By using a disposable bag as the only contact surface for the cells, the device provides excellent containment and eliminates labor intensive cleaning and sterilization. Lack of any mechanical parts except for the rocking platform dramatically reduces cost and maintenance. The gentle wave agitation provides an intrinsically low shear environment. Aeration is also performed without generating cell-damaging bubbles.

The invention is useful for animal, plant, microbial, and insect cell culture, both in free suspension as well for anchorage-dependent systems. It is very suitable for virus and pathogen cultivation because of the high degree of containment.

The bioreactor consists of a disposable pre-sterilized plastic bag that rests on rocking platform. In the preferred embodiment the platform is made of stainless steel however, the rocking platform may consist of any other rigid material such as, plastic, fiberglass, aluminum, etc.

Restraining straps prevent the bag from slipping off the platform. The inlet air pressure and outlet air pressure control will prevent over/under inflation. Other means to secure the bag such as a rigid holder, tape, or sleeve may also be used. It is critical that the bag be prevented from over inflation otherwise the bottom surface will not conform to the flat profile of the platform and poor wave action will result. It is likewise important to avoid under inflation, as an under inflated bag will have many wrinkles and will flex excessively, both of which lead to premature failure. For proper wave motion, it is critical that the bag not be completely full of liquid. In the present embodiment the liquid phase may comprise 10 to 80% of the total bag volume.

The platform may contain an integral heater controlled by a temperature sensor and controller that can be used to maintain a predetermined temperature in the cultivation chamber. The rocking action ensures that a uniform temperature is achieved in the culture fluid. Humidity of the inlet gas may be controlled to reduce evaporation. Other gases, such as carbon dioxide, may be introduced into the chamber to control pH and other environmental conditions.

Cultivation is done by inflating the bag with air, then introducing liquid media into the bag. The culture is then introduced into the bag. Rocking rate and aeration are then set at predetermined values. Samples may be withdrawn by connecting a syringe to sampling port. Virus inoculums or media additions can also be added through this port at appropriate times during the cultivation. Harvesting is done by pumping out the cell culture broth. The next batch can be initiated immediately by placing a new bag on the platform.

When bags are fixed on bioreactors and inflated with air, stress at corner is normally higher than that in area further away from corners. Two opposite bag rims are usually supported with bars and these supported bag rims are attached to the rocking plate for example by clamping down or by other means. The problem is that high corner stresses cause folding of non-bar-supported bag rims and create creases on corners. Fatigue cracking on 50 L, 200 L, and 1000 L WAVE CELLBAG™ bioreactor bags was reported in a few days to greater than 20 days of cell culture process. This may lead to scrapping of the bioreactor contents and economic loss for the user of the bioreactor. The present invention proves new solutions to minimize the formation of corner creases to significantly enhance fatigue resistance of bags.

It was found through study that formation of corner creases is also partly due to larger bag dimensions of 50 L, 200 L, and 1000 L bags than the corresponding bag holders.

A quick and easy solution is to enlarge bag holders so that bags are stretched tightly and no freedom is left for bag to fold its rims. However, this approach will alter medium flow pattern and mass transfer between air and medium inside the bag, which is not desired.

The solution to the problem according to the invention will minimize or not alter medium flow and mass transfer inside bag by alleviating corner stress or to strengthening rim of current bags by changing bag design; no bag mounting technique change is needed. In the drawings below are four examples of these new designs.

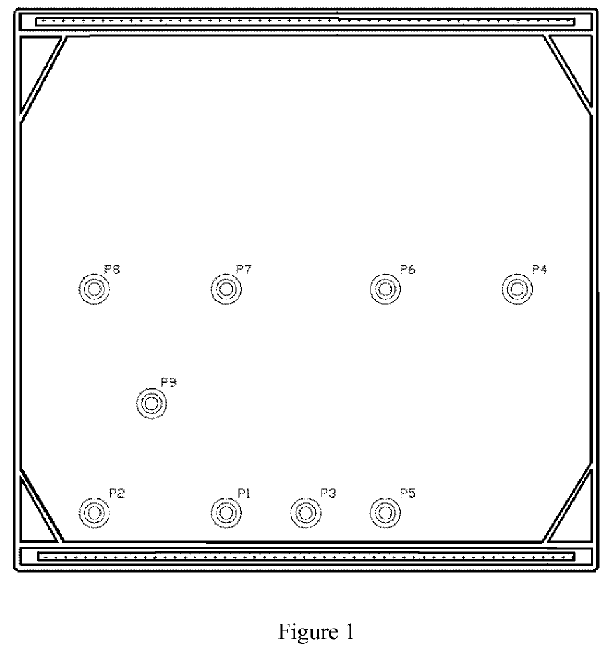

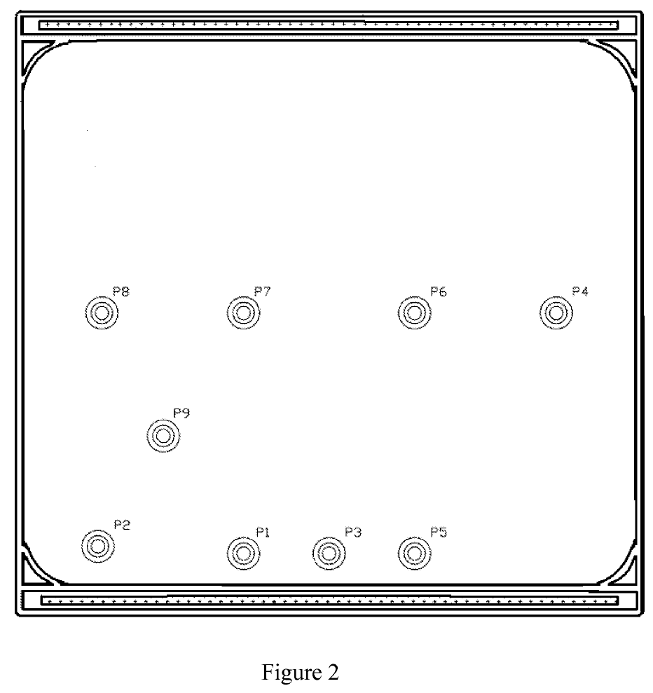

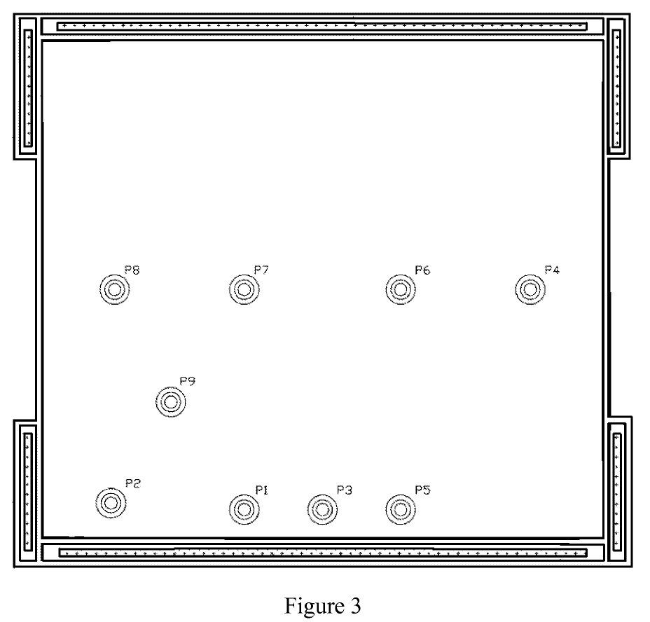

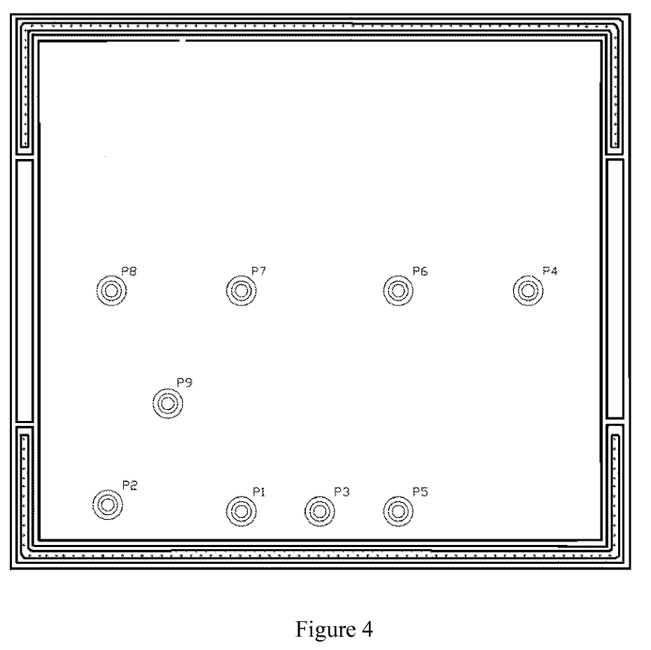

FIGS. 1 and 2 show extra structures around all corners. Besides the straight or arc seals on the corners, the seals can be any curved structure. FIGS. 3 and 4 show that two side rims are reinforced in addition to the conventional reinforcements (bars). The side rims may for example be reinforced by semi-rigid polymer rods or tubings. The strength of semi-rigid polymer rods or tubings is skilfully tuned such that the polymer rods or tubing would provide sufficient rigidity during cell culture application. These polymers must also be gamma stable. Polymers for reinforcement rod and tubings are thermoplastic or thermosetting materials, such as silicone, acrylic, nylon, polyethylene, and polyvinyl chloride (PVC), etc. Transparent flexible films for bags are single or multi-layered low density polyethylene (LDPE), linear low density polyethylene (LLDPE), ultra low density polyethylene (ULDPE), poly(ethylene-vinyl alcohol) (EVOH), polyvinylidene dichloride (PVDC), poly(ethylene-vinyl acetate) (EVA), nylon, and polyethylene terephthalate (PET). Some of these polymers might be USP class VI certified.

When bags were put on bioreactors and inflated with air and liquid, minimal (embodiments shown in FIG. 1 and FIG. 2) or no (embodiments shown in FIG. 3 and FIG. 4) corner creases were formed. Creases, if any, formed on bags according to FIG. 1 and FIG. 2, were frozen not moving with the rocking of bioreactor and hence would not develop fatigue. Bags according to FIG. 3 and FIG. 4 exhibited good and smooth contour. Several of 50 L bags of these designs showed no fatigue development in the testing duration of 40 to 50 days.

In the embodiments shown in FIGS. 1-4, bags were made of two pieces of rectangular flexible polymer films (bottom and top) via thermally sealing along the edges, which the seamless seal lines were drawn in double lines. Rigid plastic rods for bag clamping and reinforcement were drawn as a slim rectangular with many plus “+” signs, which were inserted before or after thermal seal. P1-P9 were plastic ports thermally welded to the top film of the bag. These ports provided access to the inside of the bag and could be connected to multiple assemblies for supply and exhaust of air and/or carbon dioxide, supply and exit drain of cell culture media, sampling of liquid inside bag, sensors and/or probes of PH, temperature, dissolved oxygen, dissolved carbon dioxide, and pressure, etc. Number of the ports on the bag can be more or less than 9, depending on the size and actual design of these bags.

### EXAMPLES

The present example is provided for illustrative purposes only, and should not be construed as limiting the present invention as defined by the appended claims.

### Experimental Data for Flex Fatigue Resistance

### Experiment 1

### Enhanced Flex Fatigue Resistance of 50 L WAVE CELLBAG™ at Regular Inflation

50 L WAVE CELLBAG™ was put on System 20/50 EHT WAVE BIOREACTOR™ with 20 L water inside the cellbag, which was used to simulate cyclic impact of cell culture media on cellbag. The WAVE CELLBAG™ was set to run at a temperature of 37° C., aeration of 0.15 liter per minute (1 pm), rocking speed of 26 round per minute (rpm), and a rocking angle of 7 degree. The cellbag of current commercial design had formation of creases on all corners, however, the cellbags of either corner or reinforced rim modifications showed no crease formation on the corners. Fatigue was developed on the right rear corner of cellbag of current commercial design in 6 days and leakage was developed at the same spot in 10 days, whereas no fatigue was observed on cellbags of both modifications in test duration of 28 days and 38 days, respectively. Results were listed in Table 1.

TABLE 1
Running condition and results of 50 L WAVE CELLBAG ™

| | | Time to | |
|---|---|---|---|
| Design | Test Duration | Fatigue | Fatigue Location |
| Current | 10 days | 6 days | Right Rear Top Corner, |
| Commercial | | | Leaked in 10 days |
| Design | | | |
| Triangular | 28 days | N.A | No Fatigue Observed |
| Corner | | | |
| Triangular | 50 days | N.A | No Fatigue Observed |
| Corner | | | |
| Pseudoround | 40 days | N.A | No Fatigue Observed |
| Corner | | | |
| Reinforced | 30 days | N.A | No Fatigue Observed |
| Rim | | | |
| Reinforced | 43 days | N.A | No Fatigue Observed |
| Rim | | | |
| Reinforced | 38 days | N.A | No Fatigue Observed |
| Rim | | | |

### Experiment 2

### Enhanced Flex Fatigue Resistance at Low Inflation

50 L WAVE CELLBAG™ has an air pressure of 1.4 inches water when inflated and run at regular conditions. Our experience taught us that cellbags at low inflation tended to develop fatigue at short time. Hence, the vent check valve of cellbag was removed and a flow regulator was added onto the vent. Cellbags of current commercial design, triangular corner modification, and reinforced rim modification were put on System 20/50EHT WAVE BIOREACTOR™ and connected to each other through its vent so that these bags had the same inflation. These cellbags were set to run at a temperature of 37° C., aeration of 0.25 lpm, rocking speed of 26 rpm, a rocking angle of 7 degree. The inflation pressure was kept at 0.7-0.9 inches water. Fatigue was observed on cellbag of current commercial design whereas no fatigue observed on cellbags of both modifications in test duration of 35 days.

### Experiment 3

### Enhanced Flex Fatigue Resistance of 200 L WAVE CELLBAG™ at Regular inflation

200 L WAVE CELLBAG™ was put on System 200 WAVE BIOREACTOR™ with about 100 L water inside the cellbag. The water was used to simulate cyclic impact of cell culture media on cellbag. The bioreactor was set to run at a temperature of 37° C., aeration of 1.5 to 3 lpm, rocking speed of 17 to 22 rpm, and a rocking angle of 9 degree. The cellbag of current commercial design showed fatigue at the corner, whereas cellbags of both modifications showed no fatigue in test duration. Results are listed in Table 2.

TABLE 2
Running condition and results of 200 L WAVE CELLBAG ™

| | | Test | Time to | |
|---|---|---|---|---|
| Design | Test Condition | Duration | Fatigue | Fatigue Location |
| Current | Speed: 22 rpm, | 63 days | 21 days | Left Rear Top |
| Commercial | Aeration: 3 lpm | | | Corner |
| Design | | | | |
| Triangular | Speed: 17 rpm, | 38 days | N.A | No Fatigue |
| Corner | Aeration: 2.6 lpm | | | Observed |
| Reinforced | Speed: 22 rpm, | 63 days | N.A | No Fatigue |
| Rim | Aeration: 1.5 lpm | | | Observed |

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims.

What is claimed is:

1. An inflatable bioreactor bag for cell cultivation comprised of a top and a bottom sheet of polymer material that are joined along their edges to form a sealed bag, wherein two opposing edges are formed as clamping edges to allow clamping of said bioreactor bag to a rocker type bioreactor, and wherein the bioreactor bag is provided with a wrinkle preventing structure at each end of said clamping edges.

2. The bioreactor bag of claim 1, wherein one or more of the wrinkle preventing structures is comprised of an edge joint segment that interconnects the clamping edge with an adjacent non-clamping edge, and wherein the edge joint segment connects to the clamping edge and the non clamping edge at an angle exceeding 150°.

3. The bioreactor bag of claim 1, wherein the edge joint segment is a continuous curved joint segment.

4. The bioreactor bag of claim 1, wherein the edge joint segment is an essentially straight joint segment.

5. The bioreactor bag of claim 1, wherein each wrinkle preventing structure extends more than 2% and less than 15% of the adjacent non reinforced edge, to allow unrestricted motion of the intermediate section.

6. The bioreactor bag of claim 1, wherein one or more of the wrinkle preventing structures is comprised of a reinforced section of an adjacent non-clamping edge.

7. The bioreactor bag of claim 1, wherein the reinforced section is continuously interconnected with the clamping edge at an angle.
